(19) United States
(12) Patent Application Publication
(10) Pub. No.: US 2022/0404309 A1
(43) Pub. Date: Dec. 22, 2022

(54) POTENTIOMETRIC MECHANICAL SENSORS AND TEMPERATURE SENSORS

(71) Applicant: The Regents of the University of California, Oakland, CA (US)

(72) Inventors: Ana Claudia Arias, Oakland, CA (US); Xiaodong Wu, Oakland, CA (US)

(21) Appl. No.: 17/745,383

(22) Filed: May 16, 2022

Related U.S. Application Data
(63) Continuation of application No. PCT/US2020/061351, filed on Nov. 19, 2020.
(60) Provisional application No. 62/939,523, filed on Nov. 22, 2019.

(51) Int. Cl. G01N 27/413; G01K 7/26; A61B 5/1477

(52) U.S. Cl. CPC G01N 27/413; G01K 7/26; A61B 5/1477

(57) ABSTRACT

Potentiometric sensors based on potentiometric mechanosensation and/or thermosensation mechanisms operate by regulating the potential difference between electrodes at two different electrode/electrolyte interfaces. A potentiometric sensor may include at least a first electrode, a second electrode, and a microstructured ionic hydrogel composite electrolyte in contact with the first electrode and the second electrode. Methods of making a potentiometric sensor device may include forming a first electrode on a substrate, forming a second electrode on the substrate, and applying a microstructured ionic hydrogel composite electrolyte structure in contact with both the first and second electrodes.

### CROSS REFERENCE TO RELATED APPLICATION

This Patent Application is a continuation of PCT Application No. PCT/US2020/061351, filed Nov. 19, 2020, which claims priority to U.S. Provisional Patent Application No. 62/939,523, entitled “POTENTIOMETRIC MECHANICAL SENSORS AND TEMPERATURE SENSORS,” filed Nov. 22, 2019, both of which are incorporated herein by reference in their entireties.

### STATEMENT REGARDING FEDERALLY SPONSORED RESEARCH OR DEVELOPMENT

This invention is made with government support under Grant Number 1610899 awarded by the National Science Foundation. The government has certain rights in the invention.

### BACKGROUND

As the largest organ in human body, skin plays a vital role in mediating our daily interaction with the surrounding environment. With a remarkable network of sensors, human skin can perceive external stimuli (e.g. temperature, tactile, pressure, vibration, etc) and encode the stimuli into physiological signals which are then interpreted by brain to form sensory feedback. To recreate the properties of human skin, artificial electronic skins (e-skins) have attracted tremendous attention for their promising applications in robotics, prosthetics, healthcare, and the Internet of Things. In the past decade, significant advancement has been made in this field, reporting on ultrahigh sensitivity, extremely low detection limit, multifunctionality, biocompatibility, self-healing capability, and good stretchability. Looking into these achievements, most are realized based on structure or architecture engineering (e.g. micropyramids, microcracks, wrinkling, serpentine structures, kirigami structures, etc.) and advances in materials (e.g. nanoparticles, nanowires, 2D materials, conductive polymers, biocompatible or self-healing materials, etc.). In addition to these two aspects, innovation in sensing mechanisms is an essential approach to fabricate e-skins with novel properties. Nevertheless, except for the existing sensing mechanisms (e.g. resistive, capacitive, transistive, optical, piezoelectric, triboelectric, and piezoionic), sensing mechanism innovation is rarely reported.

### SUMMARY

In a natural skin sensory system (FIG. 1A), cutaneous mechanoreceptors and thermoreceptors provide the means to perceive external mechanical and thermal stimuli via the variation in membrane potential of the skin sensory cells. At resting state, the inside of skin sensory cells is usually more negative with respect to the outside (FIG. 1B). When external stimuli are coupled with the sensory cells, the mechanically- or thermally-gated ion channels will be opened, allowing migration or flow of ions across the cell membrane. This process produces a large upswing in membrane potential (FIG. 1C, depolarization phase). With the stimuli released, the membrane potential goes back to the initial level by pumping specific ions back across the cell membrane (repolarization phase). Such mechanosensation and thermosensation mechanisms via the variation in membrane potential provides a highly-effective and energy-efficient way to perceive environmental stimuli.

According to embodiments, the skin sensory behavior may be mimicked using potentiometric mechanosensation and thermosensation mechanisms by regulating the potential difference between electrodes at two different electrode/electrolyte interfaces. Basically, when bringing two categories of rationally selected electrode materials into contact with an electrolyte, two different electrode/electrolyte interfaces can be formed with a potential difference measured between the two electrodes. Via structural and component manipulation of the electrolyte, external mechanical and thermal stimuli may be encoded into potential difference variation between the two electrodes, just like skin sensory cells coupling external stimuli into membrane potential variation. An advantage of this potentiometric sensing mechanism is that the corresponding devices do not rely on external energy input and feature ultralow power consumption (recorded less than 1 nW), which can be several orders of magnitude lower than that of conventional sensing devices. Additionally, the fabricated potentiometric mechanical sensors exhibit good capability to detect both static and low-frequency dynamic mechanical stimuli, compensating for the deficiency of self-powered piezoelectric and triboelectric devices in recording static mechanical stimuli. Additionally, an all-solution processing approach is used to fabricate high-yield potentiometric mechanical sensors and temperature sensors, e.g., using all commercially available materials, revealing good scalability and cost-efficiency.

Based on the potentiometric sensing mechanism, flexible and wearable sensing devices with novel properties are provided that are hard-to-obtain via conventional sensing mechanisms. First, a class of soft and stretchable sensors is provided, which include stretching-independent sensing performance without utilization of structure engineering strategy and expensive intrinsically stretchable conducting materials, which are two main routes to fabricate stretchable electronics. When the sensors are subjected to tensile deformation up to 50% strain, the signal output of the sensors remains highly stable (e.g., less than 1% signal variation) during the entire stretching process. Such stretching-independent sensing performance is highly desired for manufacturing fully soft, stretchable and reliable robots and prostheses. Additionally, a new type of e-skin with a single-electrode-mode configuration is provided herein, which advantageously enhances the pixel density as well as the data acquisition speed compared with traditional dual-electrode-mode e-skins based on other sensing mechanisms. These distinctive characteristics of the potentiometric sensing mechanisms also make them very attractive for future development of innovative electronic devices and smart systems.

According to an embodiment, a potentiometric sensor is provided that includes a first electrode, a second electrode, and a microstructured ionic hydrogel composite electrolyte in contact with the first electrode and the second electrode. The sensor may be a potentiometric mechanical sensor or a potentiometric temperature sensor. In certain embodiments, the first and second electrodes are located on a flexible substrate, such as a flexible polymer substrate.

In certain aspects, the first electrode comprises Ag or AgCl, and wherein the second electrode comprises a graphite carbon material, e.g., Prussian blue modified graphite carbon. In certain aspects, the microstructured ionic hydrogel composite electrolyte is sandwiched between the first electrode and the second electrode. In certain aspects, the first electrode and the second electrode are arranged in a side-by-side manner, and wherein the microstructured ionic hydrogel composite electrolyte overlays both the first electrode and the second electrode. In certain aspects, an encapsulation layer overlays or encapsulates the microstructured ionic hydrogel composite electrolyte. In certain aspects, the potentiometric sensor further includes a flexible substrate supporting one or both of the first electrode and the second electrode. In certain aspects, the flexible substrate comprises polyethylene terephthalate (PET).

In certain aspects, the microstructured ionic hydrogel composite electrolyte comprises polyvinyl alcohol (PVA), sodium chloride and glycerol (Gly). In certain aspects, a weight ratio of PVA to Gyl in the microstructured ionic hydrogel composite electrolyte is between about 0% to about 64%.

According to another embodiment, a method of making a potentiometric sensor device is provided that includes forming a first electrode on a substrate, forming a second electrode on the substrate, and applying a microstructured ionic hydrogel composite electrolyte structure in contact with both the first and second electrodes.

According to another embodiment, a flexible potentiometric sensor array is provided that includes a flexible substrate, a plurality of first electrodes on the flexible substrate, a plurality of second electrodes on the flexible substrate, and a microstructured ionic hydrogel composite electrolyte layer in contact with the plurality of first electrodes and the plurality of second electrodes. In certain aspects, the flexible potentiometric sensor array includes an encapsulation layer encapsulating the plurality of first electrodes, the plurality of second electrodes, and the microstructured ionic hydrogel composite electrolyte layer. In certain aspects, the flexible potentiometric sensor array includes control circuitry and leads connecting the control circuitry to the plurality of first electrodes and the plurality of second electrodes.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates the potentiometric mechanosensation mechanism inspired by skin sensory cells. (A) Illustrations of the cutaneous mechanoreceptors in natural skin sensory system, including Merkel cells (i), Ruffini endings (ii), Pacinian corpuscles (iii), and Meissner corpuscles (iv). (B) Schematic showing the asymmetric ion distribution across the cell membrane, forming a potential difference between the interior and exterior of the sensory cells. (C) Diagram depicting the membrane potential variation during applying a mechanical stimulus on the sensory cells (depolarization phase) and releasing the mechanical stimulus (repolarization phase). (D) Conceptual illustration of the proposed potentiometric mechanosensation mechanism. A micro-structured electrolyte is placed between two rationally selected electrode materials, with a potential difference measured between the two electrodes. When the devices are subjected to mechanical stimuli, the electrode/electrolyte interfaces would be regulated, resulting in a variation of the potential difference between the two electrodes. Through this mechanism, external mechanical stimuli can be directly coupled into voltage signal output, just like skin sensory cells coupling mechanical stimuli into membrane potential variation. (E) Diagram illustrating the response signal of the potentiometric sensors, which is analogous to the physiological signal generated by the skin sensory cells when subjected to a mechanical stimulus. The inset photograph shows a flexible and stretchable mechanical sensor based on this newly-established mechanosensation mechanism.

FIG. 2 shows creation of potential difference between electrodes at two different electrode/electrolyte interfaces. To create a potential difference for bioinspired potentiometric mechanosensation, two categories of rationally selected electrode/electrolyte interfaces are employed as shown in FIG. 2. Specifically, Prussian blue modified graphite carbon and silver/silver chloride (Ag/AgCl) are chosen as the two electrode materials and sodium chloride (NaCl) is used to prepare the electrolyte. For the Ag/AgCl electrode, abundant chloride ions (Cl⁻) are bound on the Ag/AgCl electrode surface due to the oxidation-reduction equilibrium reaction of Ag/AgCl in NaCl solution (discussed in FIG. 3). Hence, the surface of the Ag/AgCl electrode in a Cl⁻ environment is much more negatively charged compared to the Prussian blue modified graphite carbon electrode. Therefore, when bringing the Ag/AgCl and Prussian blue modified graphite carbon electrodes into contact with the NaCl electrolyte, a potential difference could be measured between the two electrodes. The potential difference is measured as an open-circuit potential. A resistor with infinite resistance (R_{∞}) is connected into the circuit and the overall resistance of the whole circuit loop is infinitely great. Hence, there is nearly no current passing through the two electrodes and the recorded potential difference could be kept constant.

FIG. 3 shows surface chemistry of Ag/AgCl electrodes in NaCl solution. Ag/AgCl is widely used as reference electrodes in the lab as well as in practical applications due to the stable potential, inexpensive construction, and non-toxic components. Ag/AgCl electrode functions as a redox electrode with a highly reversible equilibrium reaction between Ag metal and AgCl in chloride environments. The surface chemistry of Ag/AgCl electrode is based on two equilibrium reactions: the electrochemical oxidation of Ag in chloride containing solution (A) and the solubility equilibrium of AgCl (B). The overall oxidation-reduction equilibrium reaction is expressed as equation (3). For the electrochemical oxidation of Ag in a Cl⁻ environment, massive Cl⁻ ions are bound on the Ag metal surface due to the complexing interaction between Cl⁻ and Ag, which makes the Ag metal surface electrically negatively charged. On the other hand, for the solubility equilibrium of AgCl in a Cl⁻ environment, Ag⁺ ions dissolve preferentially than Cl⁻ ions, leaving a negatively charged surface. Based on these two aspects, the surface of Ag/AgCl electrode in a Cl⁻ environment is much more negatively charged than the surface of Prussian blue modified graphite carbon electrode. Hence, a potential difference could be measured between the Ag/AgCl electrode and Prussian blue modified graphite carbon electrode in NaCl electrolyte.

FIG. 4 illustrates structural and component manipulation of the electrolyte for continuous and smooth mechanosensation according to embodiments. (A-C) Schematic illustrations showing the electrode/electrolyte interfaces and the response behaviors of different prototype potentiometric mechanical sensors when bringing two electrodes (Ag/AgCl and Prussian blue modified graphite carbon) into contact with different types of electrolytes: (A) liquid ionic solution (100% NaCl solution), (B) micro-structured ionic hydrogel (≈10% polymer and ≈90% NaCl solution), and (C) micro-structured ionic composite (≈84% polymer and ≈16% NaCl solution). When the ionic solution and micro-structured ionic hydrogel are used as the electrolytes, the potential difference variation between the electrodes is too fast to manipulate, making it difficult for fabricating mechanical sensors with continuous sensing behaviors. To realize a continuous potentiometric mechanosensation process, we employ micro-structured ionic composites with controlled ion activity as the electrolyte. Via this strategy, we can successfully couple external mechanical stimuli into continuous and smooth voltage signal output of the mechanical sensors.

FIG. 5 illustrates manipulation of the ion activity of the electrolyte according to embodiments. If a salt is dissolved in an aqueous solution, the ions presented in the solution will be bounded by molecules of water, forming a hydration shell (A). The ion activity is closely relevant to the hydration degree of the ions and the relative water content in the ionic system. Hence, we are able to modulate the ion activity by tuning the water content in the electrolyte. Generally, electrolytes with higher ion activity exhibit higher ionic conductivity, thus the ion activity of the electrolytes is evaluated by measuring the conductance. Here, a polyvinyl alcohol (PVA)/NaCl/water system with varying water content is used as the electrolyte. The measured conductance of the PVA/NaCl/water system exhibits significant relevance to the water content. We extract the conductance value at frequency of 10 k Hz and plot the conductance variation against the water content (B). It is noticed that the plot of conductance vs water content can be divided into two phases. In phase i, higher conductance is observed at lower water content. This is because the ion concentration (c) becomes higher when reducing the water content in this phase, and the ion concentration plays a more important role than the ionic activity coefficient (γ) for the measured conductance. However, in phase ii, the measured conductance shows prominent decline when decreasing the water content in the system. This is due to that the reduction in water content in phase ii has a big effect on the hydration degree of the ions and significantly reduces the ionic activity coefficient. Furthermore, we measure the potential difference between the Ag/AgCl and Prussian blue modified graphite carbon electrodes in contact with PVA/NaCl/water electrolyte with varying water content (C). The measured potential difference variation at different water contents shows a similar tendency with that of the conductance variation against water content. These results verify that we can manipulate the ion activity of the PVA/NaCl/water system by tuning the relative water content, thus to regulate the potential difference between the Ag/AgCl and Prussian blue modified graphite carbon electrodes.

FIG. 6 illustrates all-solution processing fabrication of potentiometric mechanical sensors according to embodiments. (A-B) Schematic illustrations showing the mechanical sensors with sandwich structure (A) and side-by-side electrode configuration (B). (C) Force sensing behaviors of mechanical sensors with different configurations. (D) Schematic layout and photograph of the potentiometric mechanical sensors fabricated through an all-solution processing approach. See FIG. 7 for the fabrication process flow. (E) Picture of a micro-structured PVA/NaCl/Gly/water ionic composite film attached on a PET substrate. (F-G) Optical micrographs showing the created microstructure on the surface of the ionic composites via a mesh-molding strategy. See FIG. 8 for the whole process flow. Scale bar: 500 μm for F and 100 μm for G. (H) Cross-sectional morphology and surface profile of the micro-structured ionic composite. Scale bar: 500 μm.

FIG. 7 illustrates all-solution processing fabrication of potentiometric mechanical sensors according to embodiments. (A) Schematic diagram showing the all-solution processing fabrication of the potentiometric mechanical sensors using all commercially available materials. Three steps are involved: 1) stencil-printing Prussian blue modified graphite carbon and Ag/AgCl electrodes on a flexible substrate using commercial inks (i); 2) solution casting PVA/NaCl/Gly solution onto a micro-patterned template to prepare the micro-structured PVA/NaCl/Gly/water films (ii); and 3) final assembly of the potentiometric mechanical sensors (iii). See Experimental Section for the details. (B) Photograph of an array of 20 potentiometric mechanical sensors fabricated in one batch, exhibiting great scalability and cost-efficiency.

FIG. 8 illustrates scalable fabrication of micro-structured ionic composites via a mesh-molding strategy, according to embodiments. (A) Schematic diagram showing the fabrication flow of the micro-structured PVA/NaCl/Gly/water ionic composite using a mesh-molding method. First, a pre-cleaned stainless-steel screen mesh (100 mesh count) is pressed into a ceraceous template (made by stacking 5 layers of Parafilm® on a glass plate) under about 1.0 MPa for 10 min at ambient temperature. Subsequently, the screen mesh embedded in the ceraceous template is placed into a refrigerator (≈−20° C.) for 2 h, followed by quickly peeling off the screen mesh from the ceraceous template. A micro-patterned template with an inverse mesh structure is prepared. Then, PVA/NaCl/Gly aqueous solutions with desired components are cast onto the micro-patterned template. After dying the solution, micro-structured PVA/NaCl/Gly/water ionic composite films could be obtained. (B) Photograph and optical image of the screen mesh used as a template. (C) Photograph and optical image of the as-prepared micro-structured PVA/NaCl/Gly/water ionic composite film. Scale bars of the optical images in B and C are 500 μm.

FIG. 9 illustrates response behaviors of the potentiometric mechanical sensors, according to embodiments. (A) Recorded voltage signal of a mechanical sensor during gradually applying a force on the device, showing continuous and smooth mechanosensation behaviors. (B) Response behaviors of mechanical sensors with ionic composites of different Gly content. PVA/NaCl/Gly/water-X % signifies the weight ratio of Gly:PVA is X %. Gly can act as a humectant as well as a plasticizer, which enable to regulate the ion activity as well as the softness of the ionic composites. Thus, the sensing behaviors of the mechanical sensors are highly tunable by adjusting the Gly content in the ionic composites. Ionic composite of PVA/NaCl/Gly/water-32% are employed in fabrication of mechanical sensors for the following sections otherwise specified. (C) Response and recovery behaviors of the mechanical sensor when a force is applied on and released from the device. (D) Recorded voltage signal of the mechanical sensor during applying a static force on the device for 20 s. The voltage signal under the static force is nearly constant, verifying the ability of the mechanical sensor to record static mechanical stimuli. (E) Voltage signal variation of the mechanical sensor when applying a dynamic force with varying frequency (0.25, 0.5, 1.0 and 2 Hz, respectively) on the device, indicating the capability to monitor low-frequency dynamic stimuli. (F) Durability and reliability test of the mechanical sensor by loading and unloading a force on the device for 1000 cycles.

FIG. 10 illustrates a structure layout, working principle and response behaviors of potentiometric temperature sensors according to embodiments. (A) Schematic layout of the potentiometric temperature sensors fabricated through an all-solution processing approach. (B) Working principle of the potentiometric temperature sensors based on temperature-regulated ion activity of the electrolyte. (C-D) Response behaviors of the potentiometric temperature sensor when the device is immersed into warm water (35° C.) and cold water (15° C.). (E-F) Potentiometric voltage outputs and measured inner impedance of temperature sensors with different ionic composites. (G) Response behavior of potentiometric temperature sensors when they are immersed alternately into 45° C. water and 23° C. water for 20 cycles.

FIG. 11 illustrates stretchable mechanical sensors with stretching-independent sensing performance, according to embodiments. (A) Schematic diagram showing the circuit model for the operating principle of the stretching-insensitive potentiometric sensors. (B) Illustrative layout of the stretchable sensors. (C) Photographs showing the good stretchability of the sensors. (D) Recorded voltage signal of a stretchable potentiometric sensor during gradually applying a constant force onto the sensor, followed by applying and releasing 50% strain to the sensor. The insert figures give the resistance change of the elastic conductors (R₁) and voltage signal output of the sensor during stretching the sensor to 50% strain. During the stretching process, the resistance changes dramatically while the voltage signal keeps highly stable, indicating the stretching-independent sensing performance of the potentiometric sensor. (E) Measured current signal of a stretchable resistive sensor during applying a constant force onto the sensor, followed by applying and releasing 50% strain to the sensor. The current signal shows significant variation during applying/releasing 50% strain as a result of the stretching-induced interference. (F-G) Response behaviors of a stretchable tactile sensor for monitoring finger touch: without strain (F) and with 50% strain applied on the sensor (G). The measured signals of the pre-stretched and un-stretched tactile sensors are nearly identical, demonstrating that the sensor can work independently from stretching-induced interference.

FIG. 12 illustrates fabrication of stretchable mechanical sensors with stretching-independent sensing performance according to embodiments. (A) Schematic diagram showing the fabrication process flow of the stretchable sensors by combining the potentiometric mechanical sensors with elastic conductors. Stretching deformation will cause significant variation in the conductivity of the elastic conductors, while the sensor signal out is kept highly stable during the stretching process, exhibiting a stretching-independent sensing behavior. (B) Photograph of a fabricated stretchable sensor. (C) Schematic illustrations showing the structural layout of the stretchable sensor.

FIG. 13 illustrates softness, flexibility, and stretchability characteristics of stretching-insensitive sensors according to embodiments. Photographs showing a sensor is held at one end (A), bent to 180° (B), twisted to 720° (C.), rolled up (D), and stretched to 50% strain (E), respectively, showing excellent softness, flexibility, and good stretchability of this sensor.

FIG. 14 illustrates stretching interference to the sensor performance based on different sensing mechanisms, according to embodiments. Here, the sensing performance of potentiometric stretchable sensors and resistive stretchable sensors are compared when they are subjected to stretching deformation (50% strain). To normalize the comparison, the sensing elements are only changed at the sensing region (as the red dashed rectangle indicates) and all other components are kept the same (including electrodes, substrates, geometry, size, etc). Side-by-side Prussian blue modified graphite carbon electrode and Ag/AgCl electrode is used for both of the stretchable sensors. For the potentiometric sensors, a piece of micro-structured PVA/NaCl/Gly/water ionic composite is placed at the sensing region (A). In contrast, for the resistive sensors, t a piece of micro-structured conductive PDMS/CNT composite is placed at the sensing region (C). To evaluate the sensors' behaviors under stretching deformation, firstly, a constant force is loaded on the sensors to generate sensor signals. Then, the sensors are gradually stretched to 50% strain, followed by releasing the strain. The sensor signals are monitored during the stretching/releasing process. Notably, the signal output of the potentiometric sensors is highly stable (less than 1% signal variation) during the stretching/releasing process, showing a stretching-independent sensing performance (B). However, for the resistive sensors, the signal output shows prominent drifting (≈21.3% relative signal variation) during the stretching/releasing process due to the stretching interference (D). These results demonstrate the significant advantage of the potentiometric mechanosensation mechanism for constructing stretchable sensors with stretching-independent sensing performance, which is highly desired for stretchable electronics and systems.

FIG. 15 shows a comparison of electrode configurations of e-skins according to embodiments. (A) Electrode configuration of conventional dual-electrode-mode e-skins. For this electrode configuration, there must be two wires for each pixel to acquire and transport data. Spatial arrangement of the massive wires greatly limits the pixel density and pixel number of the dual-electrode-mode e-skins. (B) Electrode configuration of addressable dual-electrode-mode e-skins. For this configuration, the sensor pixels are operated one by one to avoid the interference and crosstalk. Such operating manner significantly reduces the data acquisition speed. (C) Single-electrode-mode e-skin with only one reference electrode and array of sensing electrodes. This single-electrode-mode can simplify the wire arrangement and improve the pixel density. Besides, operation of the potentiometric mechanical sensors does not involve current flow in the circuit. Hence, it is feasible to acquire data from all the pixels simultaneously with minimized interference and crosstalk between adjacent sensors/wires. These two distinct advantages make such single-electrode-mode e-skin very appealing for a wide range of applications.

FIG. 16 illustrates fabrication of single-electrode-mode e-skins according to embodiments. (A) Schematic diagram illustrating the construction process of the single-electrode-mode e-skin. First, Prussian blue modified graphite carbon and Ag/AgCl electrodes are printed on a flexible substrate. Then, thin PDMS spacers (150 μm) are placed on predefined positions, followed by pasting PVA/NaCl/Gly glue on the Ag/AgCl electrode. Finally, a micro-structured PVA/NaCl/Gly/water ionic composite film is put on the electrodes and the whole e-skin is encapsulated using a thin PDMS layer. (B) Photographs showing the assembly of the single-electrode-mode e-skin using printed electrode pattern and micro-structured PVA/NaCl/Gly/water ionic composite film.

FIG. 17 illustrates single-electrode-mode e-skin pressure mapping according to embodiments. (A) Photograph of a flexible single-electrode-mode e-skin with 6×6 pixels. (B-C) Response behaviors of the e-skin when placing a plasticine ball of ≈0.8 g (B) and a battery of ≈11.0 g (C) on the 3^{row}-3^{column} pixel. (D-F) Schematics and digital pictures showing the arrangement of steel balls (≈1.0 g) on the e-skin, forming the letter ‘C’, ‘A’, and ‘L’ (the logo letters of our university). (G-I) Spatial mapping of the potential difference of each pixel with respect to the reference electrode when the steel balls are placed on the e-skin. From the reconstructed color mapping, the letter ‘C’, ‘A’, and ‘L’ could be easily recognized, revealing the desirable pressure mapping capability of the single-electrode-mode e-skin.

FIG. 18 illustrates minimization of triboelectric interference via signal processing according to embodiments. (A) Recorded pristine signal output of a potentiometric mechanical sensor when applying static and dynamic mechanical stimuli on the device. Some spike peaks appear occasionally as a result of the triboelectric effect, which could cause interference to the sensor signals. Nevertheless, these triboelectric spikes are very sharp and can be easily filtered by subsequent signal processing. (B) Processed signal with the triboelectric spikes filtered. Such signal processing enables to minimize the triboelectric interference on the potentiometric mechanosensation process.

### DETAILED DESCRIPTION

### Potentiometric Mechanical Sensors and Temperature Sensors

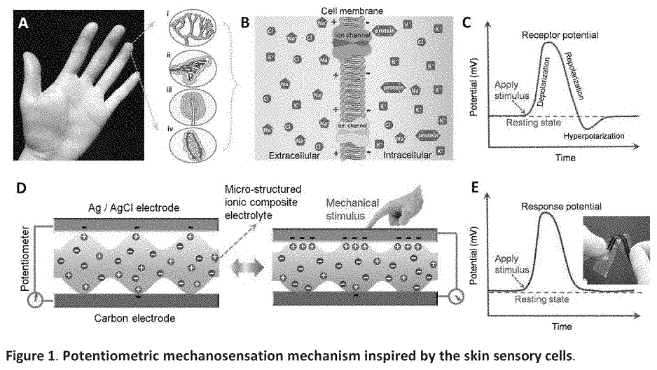

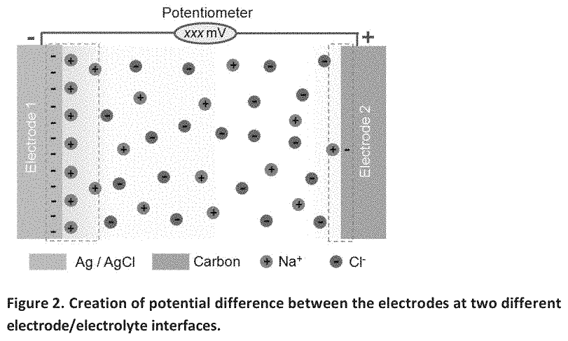

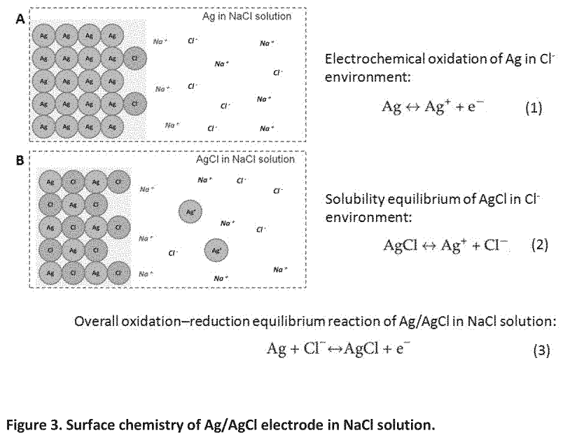

The working principle of skin sensory cells is based on the potential difference variation between two sides of the cell membrane, as illustrated in FIG. 1B-C. To imitate this process and establish a potentiometric mechanosensation mechanism, firstly, according to an embodiment, a potential difference is created between the electrodes at two different electrode/electrolyte interfaces as shown in FIGS. 2-3. By controlling or manipulating the ion activity of the electrolyte and creating microstructure on the electrolyte surface, external mechanical stimuli may be encoded into continuous potential difference variations between the two electrodes (FIG. 1D), just like skin sensory cells coupling mechanical stimuli into the membrane potential variation. This bioinspired potentiometric mechanosensation mechanism enables fabrication of soft, flexible and stretchable mechanical sensors with signal output similar to that of natural skin sensory cells (see, e.g., FIG. 1E). Moreover, such potentiometric sensors exhibit ultralow power consumption (e.g., recorded less than 1 nW), stretching-independent sensing performance, and capability to detect both static and low-frequency dynamic mechanical stimuli. These features cannot be achieved simultaneously using prior sensing mechanisms.

### Manipulation of the Electrolyte for Continuous Mechanosensation

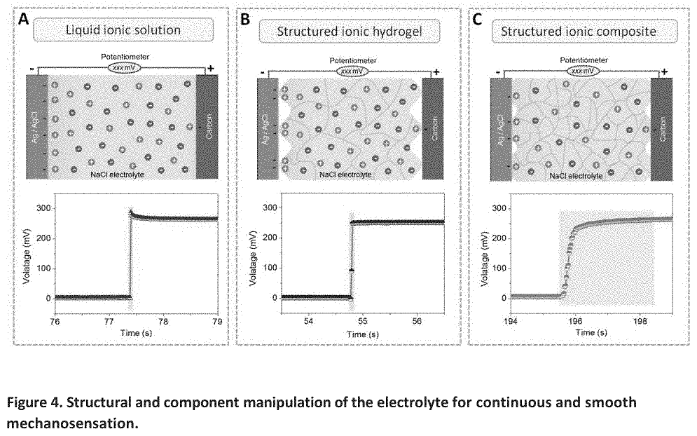

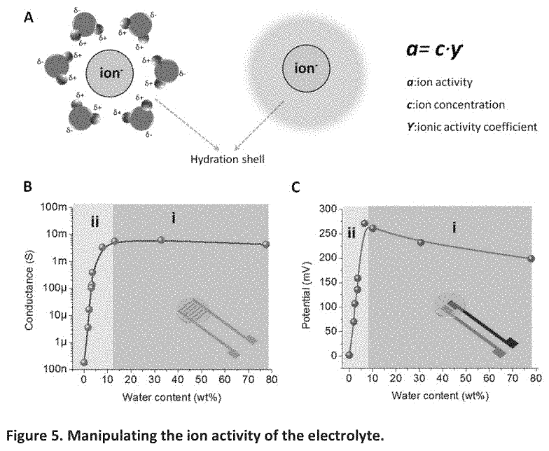

Structural as well as component manipulation of the electrolyte is important for a continuous and smooth mechanosensation process. As shown in FIG. 4A, the development of the potential difference between the two electrodes may be too fast to manipulate for a liquid electrolyte (NaCl solution). According to an embodiment, the liquid electrolyte is replaced with a solid-state and micro-structured ionic hydrogel, which enables a gradual regulation of the electrode/electrolyte interfaces (FIG. 4B). Nevertheless, when bringing the electrodes into contact with the micro-structured ionic hydrogel, the development of the potential difference between the electrodes may still be too fast to manipulate, as revealed from FIG. 4B. So, mechanical sensors based on liquid electrolyte and micro-structured ionic hydrogel have only ‘on’ and ‘off’ states and may have difficulty monitoring mechanical stimuli in a continuous way. This is because the ions in the liquid electrolyte and ionic hydrogel are fully hydrated and highly active. Even a small contact between the electrodes and electrolyte could lead to the full development of the potential difference. To achieve a continuous mechanosensation process, in an embodiment, the ion activity of the electrolyte is modulated by tuning the water content, thus to gradually regulate the potential difference between the two electrodes as shown in FIG. 5. In an embodiment, micro-structured ionic composites with tunable ion activity are used as the electrolyte, which enables smooth modulation of the electrode/electrolyte interfaces. As shown in FIG. 4C, a gradual upswing in potential difference is observed when bringing the electrodes into contact with the micro-structured ionic composites, realizing a continuous and smooth potentiometric mechanosensation process.

### All-Solution Processing Fabrication of Mechanical Sensors

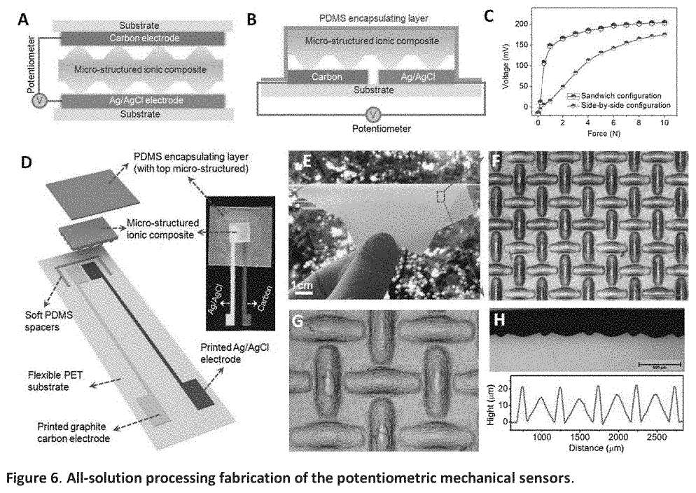

To fabricate the potentiometric mechanical sensors, in an embodiment, a micro-structured ionic composite is sandwiched between one electrode (e.g., Ag/AgCl electrode) and the other electrode (e.g., Prussian blue modified graphite carbon electrode) as illustrated in FIG. 6A. Other electrode materials may be used, such as metals or polymers or other conductive materials. Applying a force upon the mechanical sensors will increase the contact area between the ionic composite and both of the electrodes simultaneously. A larger applied force gives rise to a higher potential difference between the two electrodes, as shown in FIG. 6C. Nevertheless, this sandwich structure is not compact as there is no binding between the ionic composite and the electrodes. Additionally, it is difficult to encapsulate the mechanical sensors with this sandwich structure. To address these issues, in an embodiment, a side-by-side electrode configuration is provided with the micro-structured ionic composite placed on top of the two electrodes as illustrated in FIG. 6B and FIG. 6D. After encapsulation, e.g., with a soft polydimethylsiloxane (PDMS) layer, the mechanical sensors with such side-by-side electrode configuration are compact and also easy to construct via continuous and scalable processes. The sensitivity of the side-by-side electrode configuration is relatively lower than that of the sandwich structure (FIG. 6C), but this can be compensated by increasing the ion activity of the ionic composites. The remaining description herein will focus on the side-by-side electrode configuration unless otherwise specified.

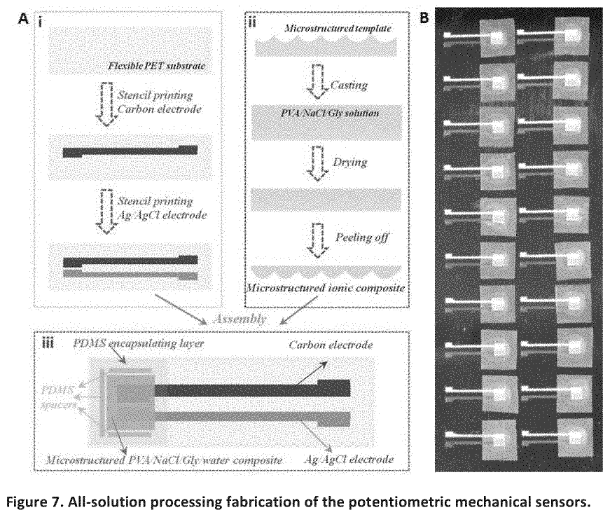

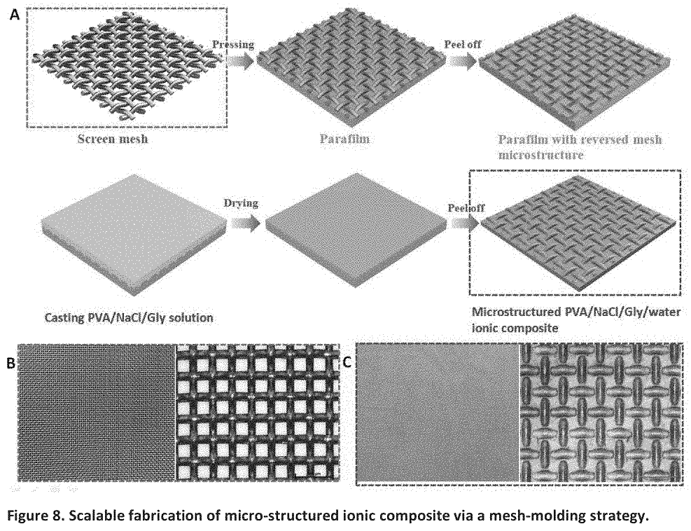

In an embodiment, an all-solution processing approach is used to fabricate the potentiometric mechanical sensors with the side-by-side electrode configuration as shown in FIG. 7A. First, the electrodes are formed on a flexible substrate. For example, commercial Prussian blue modified graphite carbon ink and Ag/AgCl ink are stencil-printed on a flexible polyethylene terephthalate (PET) substrate in sequence, followed by curing the inks into solid electrodes. For the ionic composites, in an embodiment, eco-friendly polyvinyl alcohol (PVA) is used as the polymer matrix and sodium chloride (NaCl) as the ion source. Additionally, a non-toxic glycerol (Gly) is included into the ionic composites in some embodiments. Gly can act as a humectant to tune the water content and ion activity of the ionic composites. Further, Gly serves as a plasticizer to modulate the elastic modulus of the ionic composites. With the synergy effect of these two aspects, the sensitivities of the potentiometric mechanical sensors may be easily regulated by adjusting the Gly content. The entire ionic composite may be prepared by casting PVA/NaCl/Gly aqueous solutions onto a micro-patterned template. After drying, elastic and micro-structured PVA/NaCl/Gly/water ionic composites may be obtained as shown in FIG. 6E. A mesh-molding strategy is used in an embodiment to create periodic microstructure on the PVA/NaCl/Gly/water ionic composites as shown in FIG. 8. The microstructure created via this mesh-molding strategy is very uniform and periodic, as shown in FIGS. 6F-H. Laser cutting is used in an embodiment to define the shape and size of the ionic composites. Further, the ionic composites and the printed electrodes are integrated into compact potentiometric mechanical sensors by encapsulating the devices, e.g., with a thin PDMS layer (FIG. 6D). Through this eco-friendly and economical all-solution processing approach, high-yield mechanical sensors may be fabricated (FIG. 7B), revealing good scalability and cost-efficiency.

### Sensing Performance of Mechanical Sensors

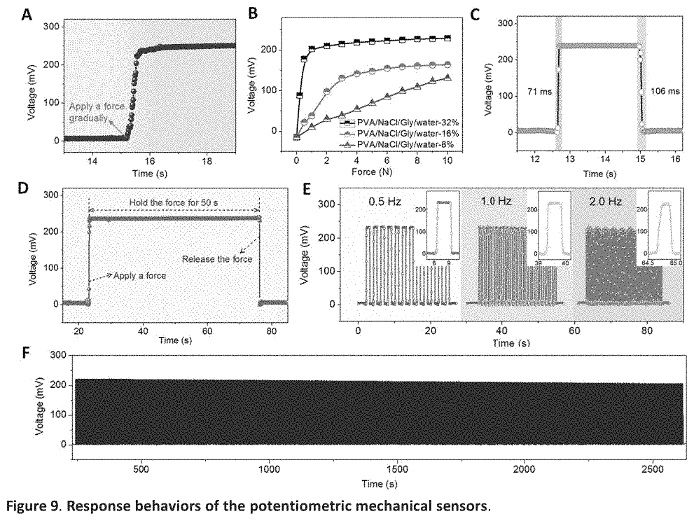

FIG. 9A shows the response behavior of a potentiometric mechanical sensor according to an embodiment when gradually applying a force on the device. The smooth increase of the voltage signal verifies the capability of the mechanical sensor for continuously monitoring external mechanical stimuli. The response performance of the mechanical sensors could be easily modulated by tuning the Gly content in the PVA/NaCl/Gly/water ionic composites. As shown in FIG. 9B, the mechanical sensor with ionic composite of PVA/NaCl/Gly/water-32% exhibits the highest sensitivity of 205.5 mV/N at the force range of 0˜1 N. Subsequently, the signal variation becomes slow (2.3 mV/N) at the force range of 1˜10 N. The mechanical sensor with ionic composite of PVA/NaCl/Gly/water-16% shows a sensitivity of 48.6 mV/N and 3.6 mV/N at the force range of 0˜3 N and 3˜10 N, respectively. For the mechanical sensor with ionic composite of PVA/NaCl/Gly/water-8%, the sensitivity tends to be constant, exhibiting 9.5 mV/N in the whole force range. The Gly content may be further tuned in the ionic composites to achieve a higher sensitivity or a broader working range, revealing great tunability of the potentiometric mechanical sensors. For example, the Gly content may range from about 0% to about 64%. This type of potentiometric sensor can find promising applications, such as for body motion detection, health monitoring, personal diagnostics, smart robotics, artificial prostheses, wearable electronic devices, human-machine interfacing, and the Internet of Things, as examples.

The response and recovery time of the mechanical sensors is measured to be ≈71 ms and ≈106 ms, respectively (FIG. 9C). Remarkably, the power consumption of the potentiometric device embodiments is ultralow and recorded less than 1 nW, which is several orders of magnitude lower than that of conventional sensing devices. This is because the measured potential difference between the two electrodes is self-generated and does not rely on the external power input. Such energy-saving mechanosensation process is highly desired for widely deployed and long-lasting sensing devices/systems.

As shown in FIG. 9D, when a static force is applied on the mechanical sensors and maintained for 50 s, the recorded voltage signal keeps nearly constant during this period. Similar to the potentiometric mechanical sensors herein, piezoelectric and triboelectric sensing devices can also generate voltage signal output, but they cannot be used to record static or slowly-varying mechanical stimuli. This potentiometric mechanosensation mechanism of the present embodiments provides a new methodology for continuous monitoring of static or slowly-varying mechanical stimuli with self-generated voltage output (as verified in FIG. 9A and FIG. 9D), compensating for the deficiency of piezoelectric and triboelectric sensing devices in this regard. Additionally, the potentiometric mechanical sensors are capable of detecting low-frequency dynamic mechanical stimuli, e.g., from 0.5 Hz to 2 Hz, as shown in FIG. 9E. The detectable frequency range of dynamic stimuli is limited by the response/recovery speed of the mechanical sensors (see FIG. 9C). Moreover, the present mechanical sensors exhibit desirable durability and reliability in cyclic test (FIG. 9F). All these capabilities make the potentiometric mechanical sensors of the present embodiments qualified as a solid platform for monitoring both static and low-frequency dynamic mechanical stimuli in an energy-efficient way.

### Potentiometric Temperature Sensors

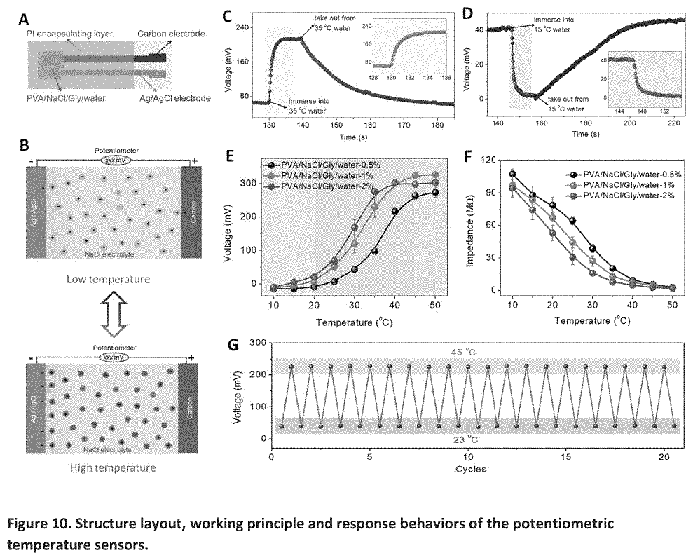

As demonstrated herein, via structural and component manipulation of the electrolyte, a potentiometric mechanosensation process may be achieved. In an embodiment, a potentiometric thermosensation process may be achieved by further manipulation of the electrolyte. To achieve this goal, the ion activity of the electrolyte may be decreased by reducing the water content to a lower level. For fabricating the potentiometric temperature sensors as illustrated in FIG. 10A, ionic composites of PVA/NaCl/Gly/water-0.5%, PVA/NaCl/Gly/water-1%, and PVA/NaCl/Gly/water-2% are used as the electrolyte, whose ion activity is lower than that of the ionic composites in the potentiometric mechanical sensors. The working principle of the potentiometric temperature sensors is based on regulation of ion activity of the electrolyte by temperature (FIG. 10B). At low temperature, the ion activity is very low and the potential difference between the two electrodes is not fully developed. By elevating the temperature, the ion activity of the electrolyte may be significantly enhanced, with a higher potential difference developed between the two electrodes. Temperature sensors based on this sensing mechanism have similar characteristics and merits with that of the potentiometric mechanical sensors.

The temperature sensing behaviors of the potentiometric thermosensation devices were evaluated. As shown in FIG. 10C, when a temperature sensor at ambient temperature (24° C.) is immersed into warm water of 35° C., the measured potential difference between the two electrodes shows a gradual yet significant increase. In contrast, when the temperature sensor is immersed into cold water of 15° C., the potential difference decreases gradually (FIG. 10D). After taking out the temperature sensor from warm or cold water, the sensor signals go back to their initial values, showing good recoverability. Remarkably, when the temperature sensor is alternately put into 45° C. water and 23° C. water for 20 cycles, the recorded signal output shows excellent reproducibility and reliability, as demonstrated in FIG. 10G.

The response behaviors of temperature sensors with different ionic composites as the electrolyte were also measured. The ion activity of the ionic composites is modulated by the tuning the Gly content in the composites. It is noticed that temperature sensors with ionic composites of higher Gly content exhibit larger output signals, as shown from FIG. 10E. This is due to that higher Gly content corresponds to higher ion activity of the ionic composites, which could facilitate better development of the potential difference between the two electrodes. To verify this, the ion activity of the ionic composites was evaluated by measuring their electrical impedance (higher ion activity generally results in lower impedance). From FIG. 10F, it can be seen that higher Gly content gives rise to higher ion activity of the ionic composites. Based on these results, the sensing behaviors of the potentiometric temperature sensors are also be highly tunable by regulating the Gly content. Temperature sensors with ionic composites of high ion activity may be more suitable to use at a lower temperature range, while sensors with low activity composites may be more suitable to use at a higher temperature range.

### Stretching-Insensitive Sensors

Compared with flexibility, stretchability will likely be a more desired characteristic for the next-generation of electronic devices and systems, as it will improve the conformability and robustness of electronic components. However, developing stretchable sensors still remains a big challenge because external stimuli cannot be measured independently from stretching-induced interference. Stretchable conductors based on structure engineering and intrinsically stretchable conductive materials are expected to exhibit small conductance changes under stretching and can be employed to alleviate this issue. Nevertheless, it is still difficult to eliminate the stretching-induced interference completely. Additionally, structure engineering usually involves sophisticated fabrication processes and is also disadvantageous for high-density device integration. For intrinsically stretchable conductive materials, complicated chemical syntheses or elaborate microphase manipulations are generally needed. These aspects limit their practical and widespread application. Hence, developing a facile and scalable approach to construct flexible sensors with stretching-independent sensing behaviors can be of great significance for creating fully stretchable electronic devices.

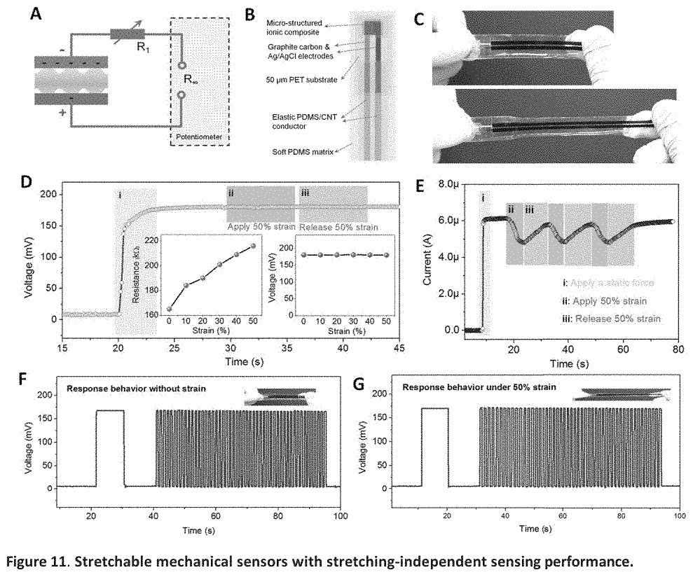

Instead of structure or material innovations, in an embodiment, a new sensing mechanism (i.e. potentiometric sensing mechanism) to fabricate stretching-insensitive sensors by combining the potentiometric sensors with elastic conductors. The operating principle of the stretchable sensors is illustrated in FIG. 11A. Basically, the potential difference between the two electrodes is measured as an open-circuit potential. For open-circuit potential measurement, a huge resistor (R_{∞}) is connected into the circuit and the overall resistance throughout the circuit is infinitely large. During stretching deformation, the resistance variation of the elastic conductors (R₁) can be neglected in comparison with the R_{∞} and will not cause substantial impact on the overall resistance of the entire circuit loop. Hence, such potentiometric sensors can work independently from stretching deformation. This strategy for fabricating stretchable sensors applies to both potentiometric mechanical sensors as well as potentiometric temperature sensors. In the following, we only test the efficiency for constructing stretchable mechanical sensors.

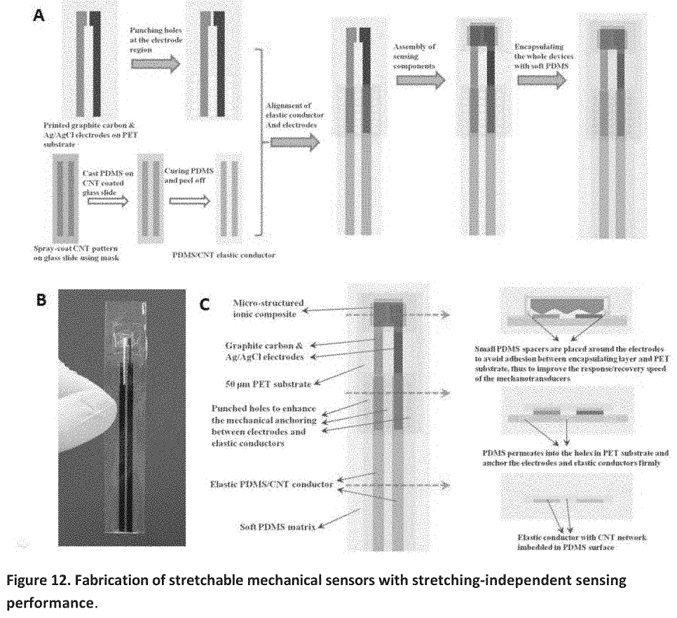

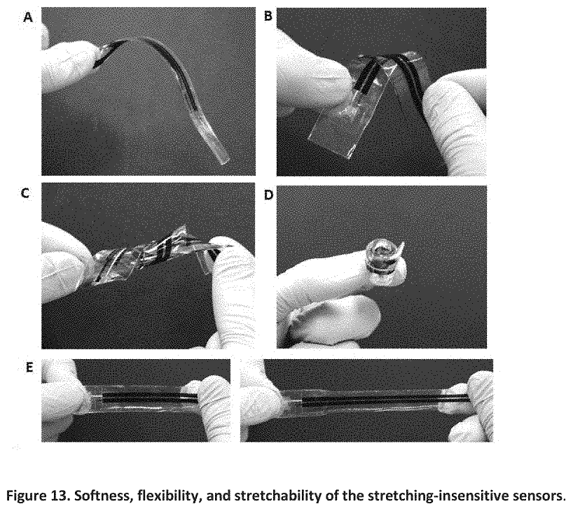

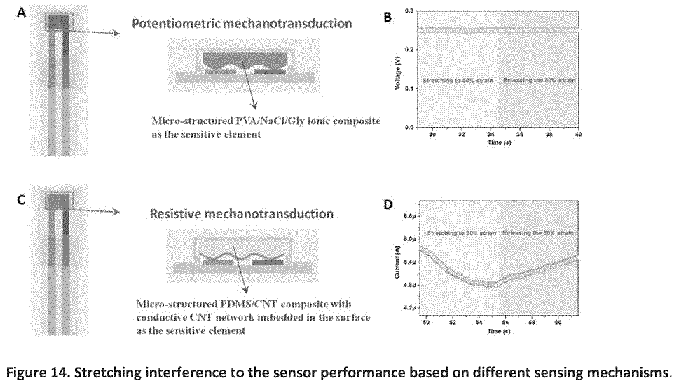

The design layout of the stretchable sensors is shown in FIG. 11B and the fabrication process is illustrated in FIG. 12. The fabricated sensors exhibit good softness, flexibility, and stretchability as presented in FIG. 11C and FIG. 13. To evaluate the impact of stretching deformation on the sensor performance, we first apply a constant force on the sensors to generate a signal output (phase i). Then the sensors are gradually stretched to 50% strain (phase ii), followed by releasing the strain (phase iii). As shown in FIG. 11D, a gradual rise of voltage signal is detected when applying a force on the sensors, indicating the desired mechanosensation behavior. During stretching the sensors to 50% strain, the measured voltage signal of the sensors keeps highly stable with nearly no signal variation (only ≈0.56% relative change) as shown FIG. 11D insert right. Nevertheless, the resistance of the elastic conductors increases remarkably during the stretching process (FIG. 11D insert left), exhibiting a relative resistance change of 31.0%. This shows that variation in resistance of the elastic conductors does not impact the sensor performance, endowing stretching-independent sensing performance to the potentiometric sensors. As a comparison experiment, we substitute the potentiometric mechanical sensor with a resistive mechanical sensor and keep all other components the same (described in FIG. 14), thus to fabricate a stretchable resistive sensor. As shown in FIG. 11E, when the resistive sensor is subjected to stretching (phase ii) and releasing (phase iii) processes, the detected signal exhibits prominent variation (≈21.3% relative change). These data verify the superiority of the potentiometric mechanosensation mechanism for constructing stretching-insensitive mechanical sensors.

As a proof-of-concept, we demonstrate the application of our stretchable sensors for perceiving finger touch independently from stretching-induced interference. First, a finger is pressed onto the tactile sensor and held for ≈10 s to apply a static force. Subsequently, the finger repeatedly presses the tactile sensor for 50 cycles to apply a dynamic force. As shown in FIGS. 11F-G, the potentiometric tactile sensors before and after stretching to 50% strain are qualified to detect both static and dynamic stimuli. Remarkably, the pre-stretched sensor (50% strain) and the un-stretched sensor (0% strain) can generate nearly identical signals (i.e. signal intensity and signal pattern). Such stretching-independent sensing capability for both static and dynamic stimulus detection is difficult to realize using conventional sensing mechanisms. We envision that these stretching-insensitive sensors are very promising for manufacturing soft robotics, stretchable prosthetics, and comfortable healthcare devices.

Single-Electrode-Mode e-Skins

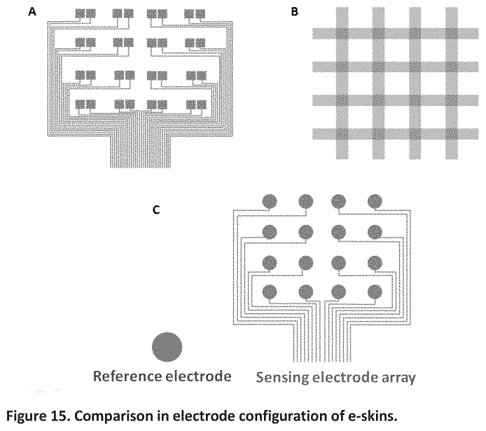

For e-skins based on resistive, capacitive and other operating mechanisms, each sensing pixel usually has two electrodes. So, there must be two wires for each pixel to acquire and transport data. Generally, such dual-electrode-mode e-skins would encounter two issues. First, based on state-of-art microfabrication techniques, spatial arrangement of the massive wires greatly limits the pixel density and pixel number of the dual-electrode-mode e-skins. Second, to avoid the interference and crosstalk between adjacent sensors/wires, the sensing pixels are usually operated one by one, which, nevertheless, reduces the data acquisition speed. These two issues pose a stumbling block for further improving the e-skins' performance. Here, utilizing the potentiometric sensing mechanism, we describe a new kind of single-electrode-mode e-skins. Basically, we use only one electrode as a reference point and measure the potential difference of other sensing electrodes with respect to this reference point (as discussed in FIG. 15). This single-electrode-mode configuration can greatly reduce the complexity of wiring and improve the pixel density of the e-skins, enabling production of more compact and sophisticated devices. Additionally, since there is no current flow involved in the potentiometric measurement, the interference and crosstalk between adjacent sensors/wires could be minimized. This makes it feasible to acquire data from all the pixels simultaneously, which could greatly enhance the data acquisition speed. This strategy for fabricating single-electrode-mode e-skins based on the potentiometric sensing mechanism applies to both mechanosensation e-skins and thermosensation e-skins. In the following, we only test the efficiency for constructing single-electrode-mode mechanosensation e-skins.

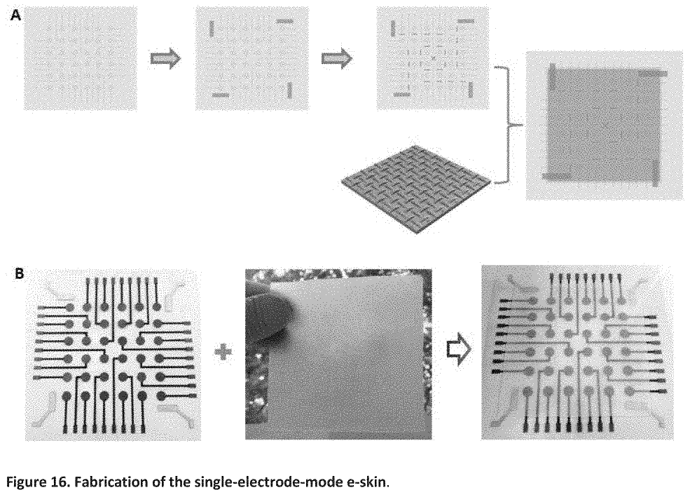

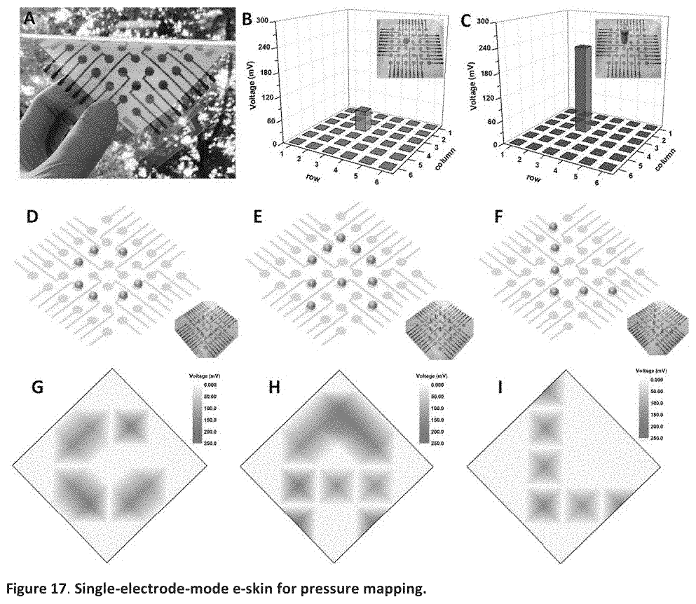

The fabrication of the single-electrode-mode e-skins is more economical compared with conventional dual-electrode-mode e-skins. Only three steps are needed (FIG. 16): 1) printing of electrode patterns on a flexible substrate, 2) solution casting of ionic composite film, and 3) final assembly of the e-skins. As a demonstration, a flexible single-electrode-mode e-skin is fabricated with 6×6 pixels (FIG. 17A, FIG. 16). The pattern and size of the e-skin could be well defined during the electrode printing procedure. To evaluate the sensing behaviors of the e-skin, a plasticine ball (0.8 g) and a battery (11.0 g) are placed on the 3^{row}-3^{column} pixel, respectively. As shown in FIGS. 17B-C, heavier object gives rise to larger potential difference between this pixel and the reference electrode, revealing good capability for differentiating the magnitude of applied force. Additionally, we arrange steel balls (≈1.0 g) into the shapes of ‘C’, ‘A’, and ‘L’ (the logo letters of our university, FIGS. 17D-F) on the e-skin and measure the potential difference of each pixel with respect to the reference electrode. As shown in FIGS. 17G-I, the reconstructed color mapping is in good consistence with distribution of the objects, demonstrating the capability of the e-skin in resolving spatial pressure distribution. With the merits of facile fabrication, ultralow power consumption, and good potential of enhancing the pixel density and data acquisition speed, such single-electrode-mode e-skins are very appealing for a wide range of applications, such as flexible touch panels, smart robots, interactive wearable devices, and so on.

In summary, potentiometric mechanosensation and thermosensation mechanisms are demonstrated. The fabricated potentiometric sensors via an all-solution processing approach exhibit ultralow power consumption (less than 1 nW), high tunability, and good capability to detect both static and low-frequency dynamic stimuli. Based on such potentiometric sensing mechanism, classes of novel devices include: 1) stretchable sensors with stretching-independent sensing performance and 2) single-electrode-mode e-skins that have good potential of enhancing the pixel density and data acquisition speed when compared with traditional dual-electrode-mode e-skins.

### All-Solution Processing Fabrication of Potentiometric Mechanical Sensors and Temperature Sensors:

A 125 μm thick PET films were used as the substrates for stencil printing of electrode patterns. Kapton tape (60 μm in thickness) films was cut into defined patterns with a laser-cutting machine and attached on the PET substrate as stencils. Prussian blue modified graphite carbon ink (C2070424P2, Gwent Electronic Materials Ltd.) was first stencil printed as one electrode using a glass slide, followed by drying at 100° C. for 10 min. Then, Ag/AgCl ink (CI-4001, Engineered Materials Systems, Inc.) was then stencil printed as another electrode, followed by curing at 120° C. for 1 hour. After removing the Kapton tape stencil, Prussian blue modified graphite carbon and Ag/AgCl electrodes were printed on the PET substrate with defined patterns. Subsequently, the surface of Prussian blue modified graphite carbon and Ag/AgCl electrodes was polished with soft and flexible wiping papers (TechniCloth, Texwipe Company) to remove the additives of the inks left on the surface, thus to obtain a more stable electrode surface.

The micro-structured PVA/NaCl/Gly/water ionic composites were prepared based on a solution casting method. Specifically, PVA/NaCl/Gly aqueous solutions containing 25 wt % PVA, 100 mM NaCl, and Gly (weight ratios of Gly to PVA were 8%, 16% and 32%, respectively.) were cast on a template with periodical microstructure molded from screen meshes (fabrication of template is described in FIG. 8). The cast PVA/NaCl/Gly solutions were first dried in fume hood at ambient temperature for 16 h and then dried at an environmental chamber at 25° C. and 50% RH for another 8 h. After drying, the PVA/NaCl/Gly/water ionic composite films (¢700 μm in thickness) were peeled off from the template and cut into defined shape and size. Subsequently, three PDMS spacers (0.15×1×5 mm³) were placed around the sensing region of the electrodes and the obtained PVA/NaCl/Gly/water ionic composites were put at the sensing region. Then, the whole mechanical sensors were encapsulated with a thin PDMS layer (≈200 μm in thickness). This outside surface of the encapsulating PDMS layer is micro-structured to avoid adhesion between the PDMS layer and external objects.

For potentiometric mechanical sensors with a sandwich structure, PVA/NaCl/Gly solution glue was pasted on the un-structured side of two pieces of ionic composite films, followed by bonding them together with the micro-structured surface outside. After drying the glue at an environmental chamber (25° C. and 50% RH) for 2 h, the ionic composite film with microstructure on both sides was sandwiched between Prussian blue modified graphite carbon and Ag/AgCl electrodes and a potentiometric mechanical sensor with sandwich structure was fabricated.

For fabrication of the potentiometric temperature sensors, Prussian blue modified graphite carbon and Ag/AgCl electrodes are printed on a flexible PET substrate as mentioned above. Subsequently, a drop of PVA/NaCl/Gly aqueous solutions (weight ratios of Gly to PVA were 0.5%, 1% and 2%, respectively.) were dropped on the sensing area of the electrodes. The PVA/NaCl/Gly solutions were first dried in fume hood at ambient temperature for 72 h and then dried at a drying oven at 60° C. for another 1 h. Finally, the potentiometric temperature sensors were encapsulated with a Kapton tape film (≈60 μm in thickness).

Fabrication of Stretchable Sensors with Stretching-Independent Performance:

Fabrication of the stretchable mechanical sensors contains three major steps. The first step is to fabricate PDMS/carbon nanotubes (CNTs) elastic conductors, as illustrated in FIG. 12. Kapton tape mask was attached onto a glass slide to define the conductive patterns, followed by spray-coating CNTs suspension (25 mL, suspended in alcohol with 1 mg/mL) on the glass slide. After removing the Kapton tape mask, PDMS precursor (the weight ratio of base to cross-linker is 10:1) was poured onto the glass slide and cured at 100° C. for 30 min. Then, the PDMS/CNTs elastic conductors were peeled off, with dense CNT conductive network imbedded in the PDMS surface. The second step is to fabricate flexible Prussian blue modified graphite carbon and Ag/AgCl electrodes on very thin PET substrate (50 μm) based on the procedure mentioned above. Three columns of holes (1 mm in diameter) were punched on the substrate near the electrodes. These holes can mechanically anchor the flexible electrodes and the elastic conductor via the permeated PDMS in the holes after final assembly process. The third step is the final assembly of the stretchable mechanical sensors. Firstly, conductive carbon paste (112-48, Creative Materials Inc.) was pasted on the Prussian blue modified graphite carbon and Ag/AgCl electrodes near the punched holes. Then, an PDMS/CNTs elastic conductor was aligned and attached to the electrodes, followed by curing the carbon paste at 100° C. for 30 min. Subsequently, PDMS spacers, PVA/NaCl/Gly/water ionic composite (PVA/NaCl/Gly/water-16%) and encapsulating PDMS layer were placed on the sensing region, respectively. Then, PDMS precursor was dropped into the holes on the substrate and the whole mechanical sensor was sandwiched between two half-cured and sticky PDMS films (prepared by curing PDMS precursor on a hotplate at 100° C. for min, followed by completely curing the device at 25° C. for 48 h (50% RH). Thus, a soft, ultra-flexible, stretchable and robust mechanical sensor with stretching-independent performance was fabricated.

Construction of Single-Electrode-Mode e-Skins:

To construct a single-electrode-mode e-skin, Kapton tape film was cut into defined patterns via laser-cutting and attached on a PET substrate as a stencil. Prussian blue modified graphite carbon and Ag/AgCl inks were stencil printed on the substrate respectively based on the procedure mentioned above. After drying the inks and removing the stencil, electrodes with defined patterns were obtained. Then, polyvinyl butyral insulating layer (10 wt %, dissolved in ethanol) was drop cast on the conductive trace, followed by placing 26 PDMS spacers (0.15×1×5 mm³) between certain electrodes (as described in FIG. 16). Subsequently, PVA/NaCl/Gly solution (weight ratios of Gly to PVA is 32%) was pasted onto the Ag/AgCl electrodes as glue and a piece of micro-structured PVA/NaCl/Gly/water ionic composite (65 mm×65 mm, weight ratios of Gly to PVA is 32%) was placed on the electrodes, with the ionic composite firmly bonded on Ag/AgCl electrode after drying the glue. Finally, a thin PDMS film (150 μm) was put on the e-skin for encapsulation.

### Characterization and Measurement:

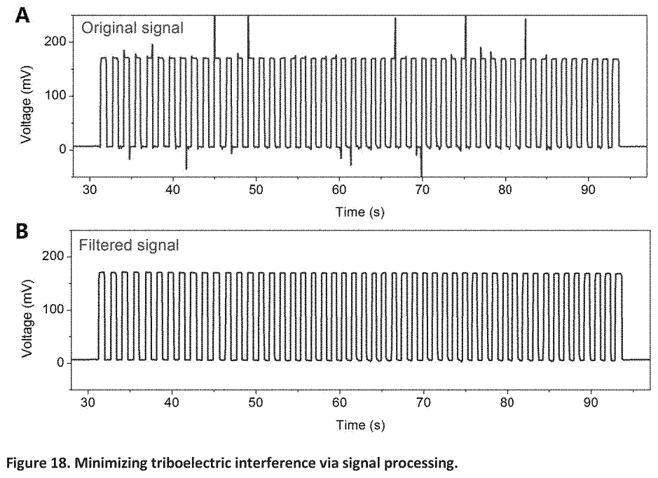

Square resistance of the Prussian blue modified graphite carbon and Ag/AgCl electrodes was measured on a four-probe resistance meter with a Keithley 2400 source meter and a four-probe stage (S-301-4, Signatone). All potentiometric voltage signals of the mechanical sensors were collected on a Keithley 2601A source meter using a voltage measure-only mode (sourcing zero current and measuring the open circuit voltage). During the potentiometric measurement, some spike signals might appear occasionally due to the triboelectric effect. However, these interferential spike signals can be easily filtered using a simple program, as discussed in detail in FIG. 18. Optical microscopic observation was conducted on an optical microscope (Eclipse 50i, Nikon). A Dektak profiler (Veeco 6M) was used for the profile measurement of the micro-structured PVA/NaCl/Gly/water ionic composite. Pressure and force measurement was conducted on a lab-built setup based on a computer controlled movable stage and a force gauge (M5, Mark-10).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Various embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this specification includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A potentiometric sensor, comprising:
a first electrode;
a second electrode; and
a microstructured ionic hydrogel composite electrolyte in contact with the first electrode and the second electrode.

2. The potentiometric sensor of claim 1, wherein the first electrode comprises Ag or AgCl, and wherein the second electrode comprises a graphite carbon material.

3. The potentiometric sensor of claim 1, wherein the microstructured ionic hydrogel composite electrolyte is sandwiched between the first electrode and the second electrode.

4. The potentiometric sensor of claim 1, wherein the first electrode and the second electrode are arranged in a side-by-side manner, and wherein the microstructured ionic hydrogel composite electrolyte overlays both the first electrode and the second electrode.

5. The potentiometric sensor of claim 4, further including an encapsulation layer overlaying the microstructured ionic hydrogel composite electrolyte.

6. The potentiometric sensor of claim 1, further comprising a flexible substrate supporting one or both of the first electrode and the second electrode.

7. The potentiometric sensor of claim 6, wherein the flexible substrate comprises polyethylene terephthalate (PET).

8. The potentiometric sensor of claim 1, wherein the microstructured ionic hydrogel composite electrolyte comprises polyvinyl alcohol (PVA), sodium chloride and glycerol (Gly).

9. The potentiometric sensor of claim 8, wherein a weight ratio of PVA to Gyl in the microstructured ionic hydrogel composite electrolyte is between about 0% to about 64%.

10. A method of making a potentiometric sensor device, the method comprising:
forming a first electrode on a substrate;
forming a second electrode on the substrate; and
applying a microstructured ionic hydrogel composite electrolyte structure in contact with both the first and second electrodes.

11. The method of claim 10, wherein the microstructured ionic hydrogel composite electrolyte structure is formed by casting polyvinyl alcohol (PVA), sodium chloride and glycerol (Gly) solutions onto a micropatterned template and thereafter drying the template to form the microstructured ionic hydrogel composite electrolyte structure.

12. The method of claim 11, wherein a weight ratio of PVA to Gyl in the microstructured ionic hydrogel composite electrolyte structure is between about 0% to about 64%.

13. The method of claim 10, wherein forming the first and second electrodes includes:
providing a flexible substrate; and
printing the first and second electrodes on the flexible substrate.

14. The method of claim 13, wherein the printing includes printing conductive ink patterns on a flexible polymer substrate.

15. The method of claim 10, further comprising encapsulating the device with a PDMS layer.

16. A flexible potentiometric sensor array, comprising:
a flexible substrate;
a first electrode on the flexible substrate;
a second electrode on the flexible substrate; and
a microstructured ionic hydrogel composite electrolyte layer in contact with the first electrode and the second electrode.

17. The flexible potentiometric sensor array of claim 16, further including an encapsulation layer encapsulating the first electrode, the second electrode, and the microstructured ionic hydrogel composite electrolyte layer.

18. The flexible potentiometric sensor array of claim 16, further including control circuitry and leads connecting the control circuitry to the first and second electrodes.

19. A flexible potentiometric sensor array, comprising:
a flexible substrate;
a plurality of first electrodes on the flexible substrate;
a plurality of second electrodes on the flexible substrate; and
a microstructured ionic hydrogel composite electrolyte layer in contact with the plurality of first electrodes and the plurality of second electrodes.

20. The flexible potentiometric sensor array of claim 19, further including an encapsulation layer encapsulating the plurality of first electrodes, the plurality of second electrodes, and the microstructured ionic hydrogel composite electrolyte layer.

21. The flexible potentiometric sensor array of claim 19, further including control circuitry and leads connecting the control circuitry to the plurality of first electrodes and the plurality of second electrodes.
